(19) United States
(12) Patent Application Publication
(10) Pub. No.: US 2025/0095819 A1
(43) Pub. Date: Mar. 20, 2025

(54) METHOD FOR PROVIDING A PERSONALIZED HEALTHCARE PARAMETER, AND HEALTHCARE MANAGEMENT SYSTEM

(71) Applicant: Roche Diabetes Care, Inc., Indianapolis, IN (US)

(72) Inventors: Samer Dajani, Carmel, IN (US); Tim Schwirtlich, Mannheim (DE)

(21) Appl. No.: 18/962,797

(22) Filed: Nov. 27, 2024

Related U.S. Application Data
(63) Continuation of application No. PCT/EP2023/064225, filed on May 26, 2023.
(60) Provisional application No. 63/365,539, filed on May 31, 2022.

(30) Foreign Application Priority Data
Aug. 31, 2022 (EP) 22 193 232.0

(51) Int. Cl. G16H 20/17; G16H 10/60; G16H 40/67

(52) U.S. Cl. CPC G16H 20/17; G16H 10/60; G16H 40/67

(57) ABSTRACT

This disclosure teaches a computer-implemented method for providing a personalized healthcare parameter to a patient and a healthcare management system supporting a more reliable and error-free determination of a personalized healthcare parameter for the patient, such as a diabetes patient. The method involves providing a healthcare application and an interface application running in a data processing component having data processors.

### RELATED APPLICATIONS

This application is a continuation of International Application Serial No. PCT/EP2023/064225, filed May 26, 2023, which claims priority to European Patent Application Serial No. 22 193 232.0, filed Aug. 31, 2022, and to U.S. Provisional Patent Application Ser. No. 63/365,539, filed May 31, 2022, the entire disclosures of all of which are hereby incorporated herein by reference.

### BACKGROUND

The present disclosure refers to a computer-implemented method for providing a personalized healthcare parameter, and healthcare management system.

In recent times, healthcare management for patients relies more and more on an integration of personalized healthcare information from different healthcare data sources. Conducting analyses of personalized healthcare data from different sources such as electronic health records (EHR) helps to improve healthcare management for the patient. On the other hand, integrating healthcare information from different sources requires reliable and safe processing of healthcare data from different sources for avoiding erroneous healthcare parameter determination which may cause, for example, non-correct diagnosis or other non-correct action such as controlling of a medical devise.

U.S. Publication No. 2021/0098133 A1 discloses a machine learning system for healthcare applications which comprises: a data ingestion pipeline configured to automatically receive patient data including stored data from an EHR database and real-time data from a plurality of data sources, the data including EHR records, claims data, and social determinants of health data; a data processing module configured to clean, extract, and process the received patient data; at least one predictive model configured to analyze the cleaned and processed data and determine a risk score for each patient; a configuration file defining the predictive model execution parameters; a tuning module configured to adjust parameters of the predictive model, including variables, thresholds, and coefficients; a retraining module configured to make further adjustments of the predictive model to remove inherent data biases; and a dashboard and reporting module configured to present the risk score to a patient care team.

WO 2021/030637 A1 refers to a patient health management platform which accesses a metabolic profile for a patient and biosignals recorded for the patient during a current time period comprising sensor data and/or lab test data collected for the patient. The platform receives patient data recorded during the current time period comprising food items consumed, medications taken, and symptoms experienced by the patient. The platform implements a machine-learned metabolic model to determine a metabolic state of the patient at a conclusion of the current time period by comparing a true representation of the metabolic state and a prediction of the metabolic state. The representation and the prediction are determined based on the recorded biosignals and the recorded patient data, respectively. The platform generates a patient-specific treatment recommendation outlining instructions for the patient to improve their metabolic state and provides the patient-specific treatment recommendation to the patient device for display to the patient.

### SUMMARY

This disclosure teaches a computer-implemented method for providing a personalized healthcare parameter and a healthcare management system supporting a more reliable and error-free determination of a personalized healthcare parameter for a patient, such as a diabetes patient.

According to an aspect, this disclosure teaches a computer-implemented method for providing or determining a personalized healthcare parameter, the method comprising: providing a healthcare application, and an interface application running in a data processing component; submitting, from the healthcare application to the interface application, a service request indicative of a request for determining a first personalized healthcare parameter, the first personalized healthcare parameter being indicative of a first healthcare condition of the patient; in response to receiving the service request, generating a first input data request by the interface application; providing the first input data request to a first service application from a plurality of service applications, wherein each of the service applications is configured to determine, for a patient, a respective personalized healthcare parameter in response to receiving respective personalized input data, and the first service application is configured to determine the first personalized healthcare parameter; receiving first input data information in the interface application, the first input data information being indicative of a data specification of first input data required by the first service application for determining the first personalized healthcare parameter; receiving the first input data information in the healthcare application; generating first personalized input data in the healthcare application, the first personalized input data being generated according to the first input data information; receiving the first personalized healthcare parameter in the healthcare application, wherein the first personalized healthcare parameter was determined in response to receiving the first personalized input data in the first service application, such determining comprising processing the first personalized input data; and outputting the first personalized healthcare parameter to a receiving device connected to the data processing component.

Another aspect refers to a healthcare management system comprising a data processing component having a plurality of data processors, the healthcare management system being configured to: provide a healthcare application, and an interface application running in the data processing component; submit, from the healthcare application to the interface application, a service request indicative of a request for determining a first personalized healthcare parameter, the first personalized healthcare parameter being indicative of a first healthcare condition of the patient; in response to receiving the service request, generate a first input data request by the interface application; provide the first input data request to a first service application from a plurality of service applications, wherein each of the service applications is configured to determine, for a patient, a respective personalized healthcare parameter in response to receiving respective personalized input data, and the first service application is configured to determine the first personalized healthcare parameter; receive first input data information in the interface application, the first input data format information being indicative of a data specification of first input data required by the first service application for determining the first personalized healthcare parameter; receive the first input data information in the healthcare application; generate first personalized input data in the healthcare application, the first personalized input data being generated according to the first input data information; receive the first personalized healthcare parameter in the healthcare application, wherein the first personalized healthcare parameter was determined in response to receiving the first personalized input data in the first service application, such determining comprising processing the first personalized input data; and output the first personalized healthcare parameter to a receiving device connected to the data processing component.

That technology proposed for the computer-implemented method and the healthcare management system supports reliable and error-free determination of one or more personalized healthcare parameters which are determined by means of one or more of the service applications. The process for correctly generating the first personalized input data assigned to the patient and providing such first personalized input data which are complying with the first input data information ensures that the first service application applied for determining the first personalized healthcare parameter is receiving correct input data. Based on such correct input data which are in line with the first input data information the input data can be correctly processed by the first service application for determining the first personalized healthcare parameter. Such reliable and error-free determination of one or more personalized healthcare parameters can be conducted even in case details about specific data processing of the first personalized input data by the service application(s) is not known. For example, even some algorithm(s) applied by the service application(s) for determining the respective healthcare parameter being not known, the correct determination of the one or more personalized healthcare parameters is ensured.

The data processing component may have one or more data processors. For example, the data processing component has a plurality of data processors. The first service application may be run on a processor different from the processor(s) on which the healthcare application and the interface application are run.

Some data requirement(s) for the first input data is collected or gathered from the first service application. In particular, the first input data request may require the first service application to inform about a first input data information. The first data information is then received from the first personalized healthcare application in the interface application and the first input data information is further received from the interface application in the healthcare application. In case determination of the first personalized healthcare parameter is requested in or by the healthcare application, the first input data information is requested in response, e.g., in real-time, thereby, making sure that, at any time the first personalized healthcare parameter/or some other personalized healthcare parameter are to be determined by the one or more of the service applications, input data are generated and provided correctly according to input data requirement(s) of the respective service application which can exchange data with the healthcare application and is requested to determine the one or more personalized healthcare parameters. For example, if some service application has received some update, such update causing change of the requirement(s) for the input data to be received by the service application, by the technology proposed, the present (or updated) data requirement(s) for the input data will always be requested by and received in the healthcare application for correctly and error-free generating the personalized input data. The input data information will be indicative of such requirement(s).

The interface application may also be referred to as a generic interface (application) between the healthcare application and the plurality of service applications. The generic interface is ensuring or providing for that respective input data are submitted to the service applications according to the respective input data requirement(s) indicated by the input data information gathered by the healthcare application prior to actually submitting input data to the service application(s).

The generating of the first input data request may further comprise, in the interface application, determining one or more pre-selected first service applications from the plurality of service applications, each of the one or more pre-selected first service applications being configured to determine the first personalized healthcare parameter; and selecting the first service application from the one or more pre-selected first service applications. Prior to receiving the service request indicative of the request for determining the first personalized healthcare parameter from the healthcare application, by the interface application one or more pre-selected first service applications may be determined, each of such pre-selected first service applications being configured to determine the first personalized healthcare parameter. A plurality of service applications may be available for determining the first personalized healthcare parameter. The service applications form the plurality of service applications may require different input data specification. In this example, the interface application is pro-actively gathering information about the first service applications in advance (prior to receiving the service request), and, therefore, is ready to select the first service application if an actual service request is received in the interface application. The interface application may store information about the one or more pre-selected first service applications in a local memory accessible by and assigned to the interface application. The information about the one or more pre-selected first service applications may be updated by the interface application, for example, after a pre-determined time limit has lapsed since the one or more pre-selected first service applications have been determined last time.

The computer-implemented method may further comprise determining, by the interface application, the one or more pre-selected first service applications from at least one of: a first plurality of service applications in a local database locally accessible by and assigned to the interface application; and a second plurality of service applications remotely accessible in a remote database. The plurality of service applications may be provided for access by the interface application locally and/or remotely. For example, access to a remote database may be provided via internet. A service provider may provide a service application homepage which offers a plurality of service applications for use. Other or additional service applications may be accessible by the interface application in a local database assigned to one or more data processors on which the interface application itself is running or implemented.

The provider of the healthcare application (and optionally the interface application) can be different from the provider(s) of at least a subset or all of the plurality of service applications. In one example, it is not known by the provider of the healthcare application (and interface application) which algorithm(s) are actually applied for determining the respective healthcare parameter(s) by one or more of the service applications.

The submitting of the service request may further comprise providing service application information with the service request, the service application information being indicative of a selection of the first service application; and, in response to the service application information, generating the first input data request by the interface application, and providing the first input data request to the first service application. In this example, the service request provided from the healthcare application to the interface application is indicative of the selected first service application which is to be requested to determine the first personalized healthcare parameter. For example, the healthcare application may have been provided with general information about service applications available for use or invoking. Based on such information the service request may be provided to contain the service application information indicative of the selection of the first service application which may be one from a plurality of service applications all configured to determine the first personalized healthcare parameter and “known” to the healthcare application. In response to such type of service request containing the service application information, the interface application will provide the first input data request to the selected first service application. In this example the healthcare application itself at least in part has control about which service application is requested to determine the first personalized healthcare parameter. Alternatively, the healthcare application may just submit the input request to the interface application (without identifying a preferred or selected service application). In response, the interface application will determine or select the service application which is requested to actually determine the first personalized healthcare parameter.

The generation of the first personalized input data may further comprise, in the healthcare application, receiving healthcare data from one or more healthcare data sources. The healthcare data sources may be selected from the following group of healthcare data sources: unstructured healthcare database, clinical healthcare database, and regulated healthcare device data; and generating the first personalized input data from the received healthcare data. With respect to the unstructured healthcare database, for example, it may provide for personalized fitness data of the patient. The clinical healthcare database, for example, may provide for personalized electronic health record (EHR). With respect to regulated healthcare device data, for example, measurement data from a blood glucose meter and/or an insulin pump assigned to the patient may be provided. In an example, alternatively or in addition, the healthcare application may be receiving healthcare data not from a database, but from some other healthcare data source such as a personal fitness device collecting personalized fitness data, and/or the blood glucose meter collecting measurement data for the patient. Thus, the healthcare data may be received in the healthcare application directly form one of such devices and/or through one of the healthcare databases after storing the healthcare data in such database before.

One or more of the healthcare applications may be configured to semantically recognize which data or data points are needed from the input data and semantically collect matching data or data points. A prioritization may be applied for data for the same input data point from multiple healthcare data sources. A step for interpolating and/or estimating missing data (points) from existing information may be applied. For example, a body mass index (BMI) may be determined for the patient from information on body weight and height for the patient. Alternatively or in addition, waist size may be determined for the patient from information on body weight and height for the patient.

When checking whether the received healthcare data are complying with the data specification of first input data of the first service application non-compliance is determined, the generation of the first personalized input data from the received healthcare data may further comprise converting at least some of the received healthcare data having or being provided with (according to) a source data specification into the first personalized input data having or being provided with the data specification of first input data. Because of receiving the first input data information being indicative of the data specification of first input data required by the first service application in the healthcare application (e.g., from the interface application), the healthcare application can check or decide whether the healthcare data received from the data source are complying with the data specification of first input data of the first service application or not. In some cases, a first data conversion may be applied for converting the received healthcare data being provided with or according to a source data specification which is different from the data specification of first input data into the first personalized input data complying with the data specification of first input data. The source data specification is defining a specification for the source data. Providing the source data with or according to the source data specification refers to providing source data which are in line with or fulfilling such specification, such as format of data. For example, the first data specification of input data may define or specify in which respective unit(s) of value the first input are required to be received by the first service application. The first data specification of input data may define or specify some specific unit of value for some of the healthcare data. If the healthcare data received from the data source do not comply with such unit of value, conversion is applied in the healthcare application.

The generation of first personalized input data may particularly comprises receiving healthcare data from one or more healthcare data sources and generating the first personalized input data from the received healthcare data which includes checking whether the received healthcare data are complying with the data specification of first input data of the first service application.

The computer-implemented method may further comprise, in the healthcare application: converting the first personalized healthcare parameter having an output data specification to the first personalized healthcare parameter having a first source data specification required by a first healthcare data source from the group of the healthcare data sources; and storing the first personalized healthcare parameter having the first source data specification in the first healthcare data source. The first personalized healthcare parameter determined by the first service application may be provided or returned to one of the healthcare data sources, thereby, for example, storing a present or updated value for the first personalized healthcare parameter in the healthcare data source. Such storing can provide for updating an electronic healthcare record for the patient.

The submitting of the service request may further comprise generating the service request in the healthcare application in response to a trigger event detected in the healthcare application. In this example, the service request for determining the first personalized healthcare parameter is initiated after the trigger event has been detected in the healthcare application. The generating of the service request in the healthcare application in response to the detection of the trigger event may be conducted automatically in the healthcare application, for example, in real-time in response to the detection of the trigger event.

The (e.g., first) trigger event may be indicative of a user input received in the healthcare application. For example, user input may indicate request for updating the electronic health record for the patient with respect to the first personalized healthcare parameter. In example, a physician may request determination of the first personalized healthcare parameter for diagnostic reason. Alternatively or in addition, a device input may be received in the healthcare application, for example, from some medical device, wherein the device input is indicative of a request from the medical device for determining the first personalized healthcare parameter which following may be applied for controlling operation of the medical device.

The (e.g., second) trigger event may be indicative of a trigger point in time. For example, a time limit may be set or pre-determined. After the time limit has lapsed a trigger point is detected in the healthcare application, thereby, initiating generation of service request. The time limit may be set or pre-determined, for example, by a user input.

The (e.g., third) trigger event may be indicative of a parameter value change for a medical parameter applied for determining a former first personalized healthcare parameter by means of the first service application, thereby, updating the former first personalized healthcare parameter with the first personalized healthcare parameter. Updating of the former first personalized healthcare parameter comprises newly calculating or determining the first personalized healthcare parameter by the first service application. For example, the medical parameter may refer to a blood glucose level of the patient, and the blood glucose level has been applied as an input parameter for determining the first personalized healthcare parameter before. Because of a change of the blood glucose level (presently) detected, newly determining the first personalized healthcare parameter is triggered, such determining of the first personalized healthcare parameter taking into account the different (present) blood glucose level caused by the change. A threshold value may be pre-determined for the medical parameter, for example, by user input. The determining of the first personalized healthcare parameter may only be triggered if the medical parameter is reaching or crossing threshold value. For example, the determining of the first personalized healthcare parameter may be triggered if the medical parameter is reaching or crossing a value change of 10 percent, preferably a value change of 20 percent, and more preferably a value change of 30 percent compared to the value determined for the first personalized healthcare parameter before.

The computer-implemented method may further comprise: receiving the first personalized input data by the interface application; verifying, based on the first input data information received in the interface application, whether the first personalized input data are complying with the first input data information by the interface application; and providing the first personalized input data from the interface application to the first service application, only if the first personalized input data are verified to comply with the first input data information. In this example, there is some check (e.g., plausibility check) for whether the first personalized input data received by the interface application from the healthcare application are complying with the first input data information prior to providing the first personalized input data to the first service application by the interface application. For example, the plausibility check may provide for checking whether the first personalized input data are within (or outside of) some value or data range which may be pre-defined. Such optional measure or check further improves safe and correct use of the first service application for determining the first personalized healthcare parameter.

The outputting of the first personalized healthcare parameter may further comprise receiving the first personalized healthcare parameter by a medical device; and controlling operation of the medical device according to the first personalized healthcare parameter. For example, the first personalized healthcare parameter may be applied for controlling at least one of a blood glucose meter, a blood glucose sensor, and an insulin pump. In this or other embodiments, the determining of the first personalized healthcare parameter may be conducted or applied for a diabetes patient. With respect to the controlling of the blood glucose meter, in response to receiving the first personalized healthcare parameter, some measurement for detecting present blood glucose level of the patient may be initiated. In another example, some new or additional analysis of measurement data indicative of the blood glucose level of the patient may be conducted in response to providing the first personalized healthcare parameter, wherein the first personalized healthcare parameter may be crossing some threshold value for the first personalized healthcare parameter. Because of such threshold crossing, the new or additional analysis of the measurement data may be started. Similarly, detecting present measurement data by the blood glucose sensor and/or administrating an insulin bolus by the insulin pump may be initiated or started in response to providing the first personalized healthcare parameter.

The outputting of the first personalized healthcare parameter may further comprise at least one of, for the first personalized healthcare parameter, displaying on an output display device and printing by an output printer device.

The plurality of service applications may comprise service applications configured to determine, for the patient, one or more personalized healthcare parameters selected from the following group: risk prediction, therapy management, and treatment recommendation. With respect to risk prediction, a likelihood of chronic kidney disease may be determined by at least one of the service applications. Regarding therapy management and/or treatment recommendation, one or more personalized healthcare parameters selected from the following group may be determined by the service application(s): blood glucose level, insulin dose, amount of carbohydrate intake, recommended time for insulin administration or carbohydrate intake.

A further or second service request may be initiated, such as automatically triggered, in the healthcare application in response to receiving and/or outputting the first personalized healthcare parameter. The further service request is indicative of a further or second request for determining a second personalized healthcare parameter, the second personalized healthcare parameter being different from the first personalized healthcare parameter and indicative of a second healthcare condition of the patient. Steps conducted in response to the further service request will be similar or comparable to the steps conducted in response to the first service request. A second service application (which may be different from the first service application) will provide for determining the further personalized healthcare parameter. The second service application will receive second input data according to second input data information. For example, if the first personalized healthcare parameter is indicative of kidney disease risk for the patient, a risk of liver disease may be determined by the further personalized healthcare parameter. Such determining of at least one additional or further personalized healthcare parameter, for example, may be triggered in response to the first personalized healthcare parameter reaching or crossing some threshold value. For example, if the risk for kidney disease may be determined being high for the patient, determination of the risk of liver disease may be initiated in response to such finding in the healthcare application.

With respect to the healthcare management system, the embodiments described above for the computer-implemented method may be provided mutatis mutandis.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a schematic representation of healthcare management system provided with a data processing component or system having a plurality of data processors, and a software component comprising a plurality of software applications running on the plurality of data processors;

FIG. 2 is a schematic representation of an arrangement for performing a computer-implemented method for determining or providing a personalized healthcare parameter in the healthcare management system; and

FIG. 3 is a schematic representation for a computer-implemented method for determining a personalized healthcare parameter.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

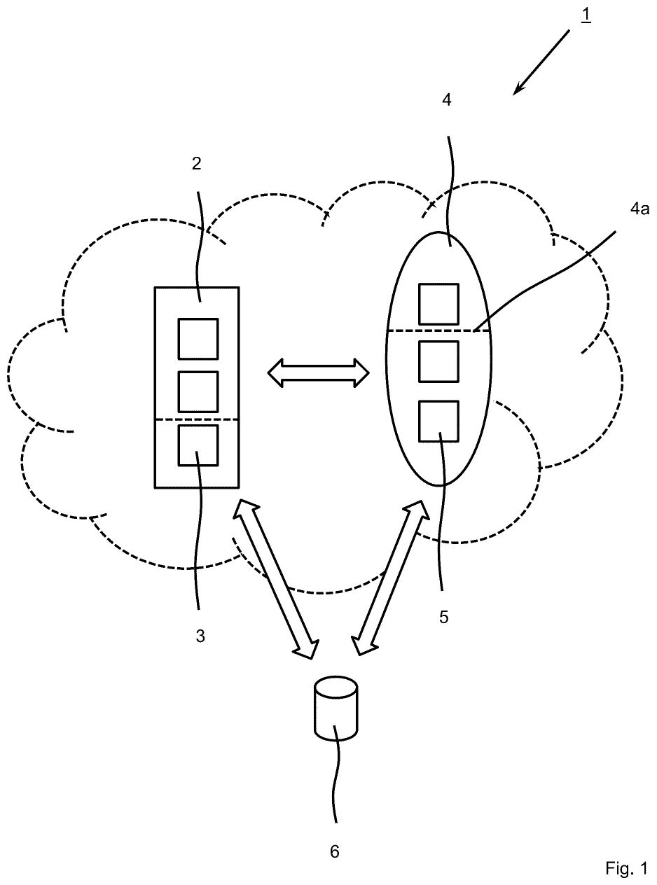

FIG. 1 shows a schematic representation of a healthcare management system 1 provided with a data processing component 2 having a plurality of data processors 3, and a software component 4 comprising a plurality of software applications 5 implemented or running on the plurality of data processors 3. Further, the arrangement provided with the data processing component 2, and the software component 4 is functionally connected to at least one data source or database 6 accessible by at least one of the data processing component 2 and the software component 4 and configured to provided healthcare data for one or more patients. Alternatively, the database 6 may be integrated with the arrangement provided with the data processing component 2, and the software component 4. Hardware and/or software elements or components of the healthcare management system 1 may be provided in one or more computer network systems. Data processors from the plurality of data processors 3 may be located in different (separated) locations schematically indicated by dashed line 4a in FIG. 1. Similarly, software applications from the plurality of software applications 5 may be running on different (separated) data processors. Exchange of data may be conducted by wireless and/or wired data transmission.

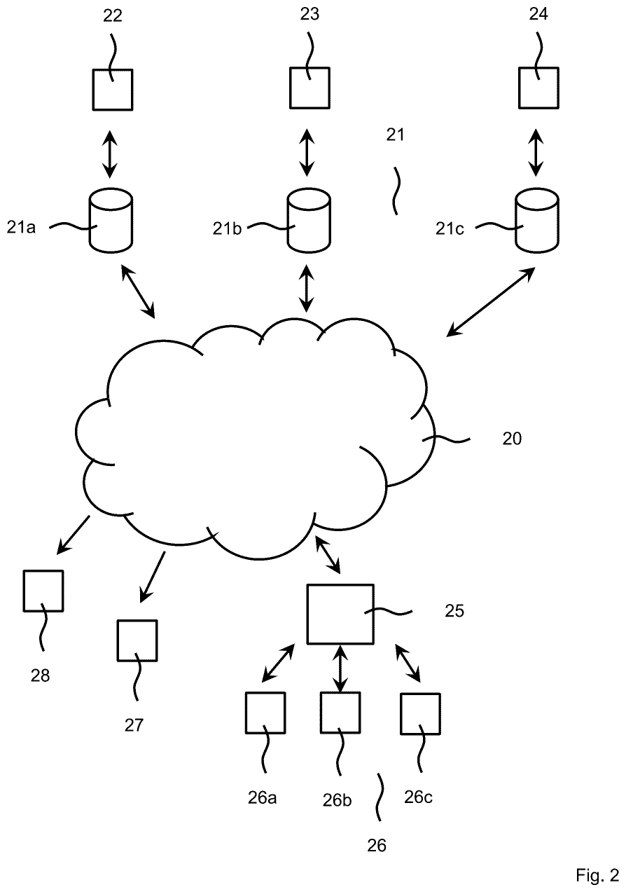

FIG. 2 shows a schematic representation of an arrangement comprising elements or modules provided, for example, by the healthcare management system 1 for performing a computer-implemented method for providing or determining a personalized healthcare parameter. In general, the arrangement of FIG. 2 may be provided based on the healthcare management system 1. On the data processing component 2 at least some of the plurality of software applications 5 are running, wherein each of the applications is configured to provide one or more functionalities of the healthcare management system 1 applied in the computer-implemented method for determining a personalized healthcare parameter.

Referring to FIG. 2, a healthcare application 20 is implemented by one or more of the software applications 5 of the software component 4. The healthcare application 20 is configured to extract healthcare information from healthcare data and provide insights into a patient's healthcare conditions, for example, for allowing diagnosis based on the healthcare condition. The healthcare application 20 may be a personalized healthcare application assigned to and personalized for a patient. Personalization may be implemented by receiving personalized information such as name, birthday and/or personal medical data of the patient in the healthcare application 20 and providing restricted access to personalized healthcare data based on such personalized information only.

By the healthcare application 20 a plurality of healthcare data sources or resources 21 is accessible. The healthcare data sources 21 may be implemented by a plurality of databases accessible by the healthcare application 20. A first healthcare data source 21a may be provided as an unstructured healthcare database containing, for example, personalized fitness data gathered by a fitness device 22 assigned to or used by the patient. A second healthcare data source 21b provides for clinical healthcare data such as personalized electronic health record (EHR) received via user input 23. The second healthcare data source 21b may provide healthcare data complying with the FHIR Standard (FIHR—Fast Healthcare Interoperability Resources). The FHIR specification such as HL 7 FHIR describes a set of resources and several different frameworks for exchanging data resources between different healthcare-related systems.

According to the example shown in FIG. 2, there is a third healthcare data source 21c providing regulated healthcare device data such as measurement data from a medical device 24, for example, a blood glucose meter and/or an insulin pump assigned to the patient.

The healthcare application 20 is configured to receive and process healthcare data from one or more of the different healthcare data sources 21. Also, the healthcare application 20 may be configured to provide healthcare data to the healthcare data sources 21 and provide such healthcare data for storing in one or more of the healthcare data sources 21. For conducting such data generation for data storage, the healthcare application 20 may be configured to generate healthcare data complying with a source data specification specifying characteristics of the data storable in at least one of the healthcare data sources 21a, 21b, 21c.

According to FIG. 2, an interface application 25 is provided which is configured to exchange electronic data with the healthcare application 20. The interface application 25 may be running on the same subset of data processors like the healthcare application 20, or on a different subset of data processors from the data processing component 2. The interface application 25 provides for a generic interface between the healthcare application 20 and a plurality of service applications 26. The plurality of service applications 26 may be implemented by one or more of the software applications 5. The one or more software applications implementing the plurality of service applications 26 may be separated from the one or more software applications providing for the healthcare application 20. Each of the service applications 26a, 26b, 26c is configured to determine, in response to receiving (e.g., personalized) input data, one or more personalized healthcare parameters indicative of a healthcare condition for the patient for whom the input data have been gathered before. For example, the plurality of service applications 26 may be configured to determine one or more personalized healthcare parameters for the patient selected from the following group: likelihood of chronic kidney disease, blood glucose level, insulin dose, amount of carbohydrate intake, and recommended time for insulin administration or carbohydrate intake.

In order to correctly determine the respective personalized healthcare parameter, each of the service applications 26a, 26b, 26c from the plurality of service applications 26 will require to receive input data according to an individual input data specification. Such input data specification, for example, may refer to input data parameters such as name, data type (string, object, array), and/or some unit of value (such as milliliter, gram, kilogram, . . . ). The respective service application from the plurality of service application 26 will determine one or more personalized healthcare parameters correctly only if the input data received are complying with the input data specification of such service application.

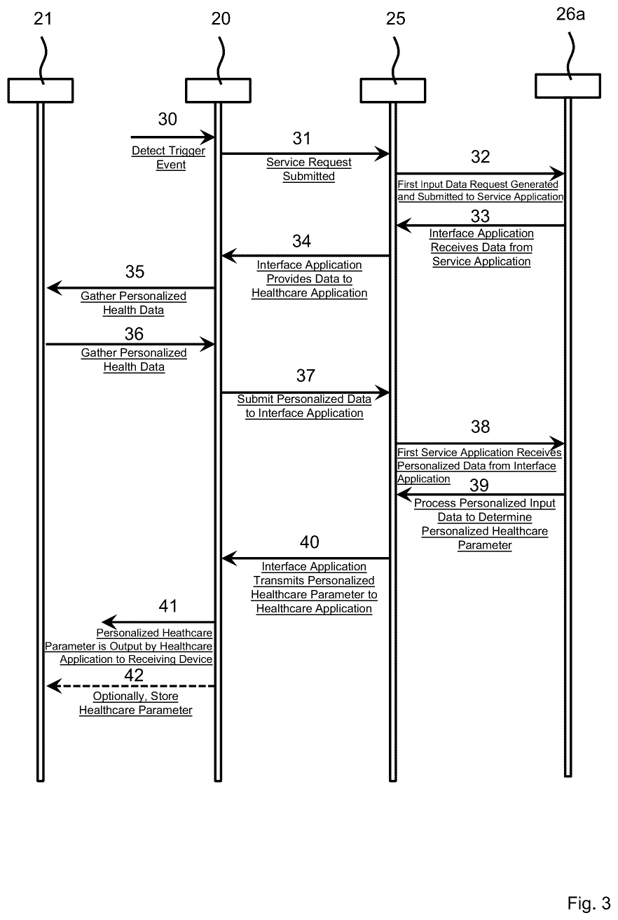

FIG. 3 shows a schematic diagram of steps conducted in computer-implemented method for determining a personalized healthcare parameter for the patient in the arrangement of FIG. 2.

In step 30, a trigger event is detected in the healthcare application 20. For example, user input may be received as a trigger event. Alternatively, some time limit may have lapsed for triggering the event. The event trigger refers to a request for determining a first personalized healthcare parameter for the patient. The trigger event such as user input may be indicative of personalized patient information such as name, birthday and/or personal medical data. Alternatively, some personalized code assigned to the patient may be provided with or in response to the trigger event in the healthcare application 20. For example, a device code assigned to a medical device such as a blood glucose meter which in turn is assigned to the patient may be indicative of the patient.

In step 31, a service request indicative of the request for determining the first personalized healthcare parameter is submitted from the healthcare application 20 to the interface application 25, after the service request has been generated by the healthcare application 20 in response to detecting the trigger event. The first personalized healthcare parameter to be determined is indicative of a first healthcare condition of the patient.

In response to receiving the service request by the interface application 25, a first input data request is generated by the interface application 25 and submitted to a first service application 26a (step 32). The first input data request will require the first service application 26a to inform about a first input data information indicative of a data specification for first input data required to be received by the first service application 26a for determining the first personalized healthcare parameter. According to step 33, the interface application 25 receives data or information indicative of the first input data information from the first service application 26a. After receiving the first input data information, the interface application 25 will provide data or information indicative of first input data information to the healthcare application 20 (step 34).

In response to receiving the first input data information, the healthcare application 20 will gather personalized health data from one or more healthcare data sources from the plurality of healthcare data sources 21 (steps 35, 36). Based on such healthcare data gathered or collected, the healthcare application 20 will generate first personalized input data which are complying with the first input data information. Such first personalized input data are submitted to the interface application 25 in step 37.

The generating of the first personalized input data from the received healthcare data may comprise applying a first conversion of data specification by converting at least some of the received healthcare data having a source data specification to the first personalized input data having or being in line with the first input data information. Because of receiving the first input data information in the healthcare application 20 (e.g., from the interface application), the healthcare application 20 can check or decide whether the healthcare data received from the data source(s) 21 are complying with the data information provided for the input data of the first service application 26a or not. In some cases, a first data conversion may be applied for converting the received healthcare data being provided with a data source specification which does not comply with the first input data information into the first input data complying with the first input data information. For example, the first input data information may define or specify some specific unit of value for some of the healthcare data. If the healthcare data received from the data source(s) 21 do not comply with such unit of value, with respect to such unit of value conversion is applied in the healthcare application 20.

The first service application 26a will receive the first personalized input data from the interface application 25 (step 38). After processing the first personalized input data for determining the first personalized healthcare parameter by the first service application 26a in step 39, the first service application 26a will return the first personalized healthcare parameter determined to the interface application 25. Following, in step 40 the interface application 25 will transmit the first personalized healthcare parameter to the healthcare application 20. The first personalized healthcare parameter will be outputted by the healthcare application (step 41) to a receiving device receiving the first personalized healthcare parameter.

For example, the first personalized healthcare parameter may be provided to an output device 27 for printing and/or displaying the first personalized healthcare parameter. Alternatively or in addition, the first personalized healthcare parameter may be provided by the healthcare application 20 to a medical device 28 assigned to the patient such as a blood glucose meter or insulin pump. Operation of such medical device 28 can be controlled according to or in dependence on the first healthcare parameter.

Optionally, the first healthcare parameter may be stored in one of the healthcare data sources 21 in step 42.

While exemplary embodiments have been disclosed hereinabove, the present

invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method for providing a personalized healthcare parameter to a patient, comprising:
providing a healthcare application and an interface application running in a data processing component;
submitting, from the healthcare application to the interface application, a service request indicative of a request for determining a first personalized healthcare parameter, the first personalized healthcare parameter being indicative of a first healthcare condition of the patient;
in response to receiving the service request, generating a first input data request by the interface application;
providing the first input data request to a first service application from a plurality of service applications, wherein
each of the service applications is configured to determine, for the patient, a respective personalized healthcare parameter in response to receiving respective personalized input data, and
the first service application is configured to determine the first personalized healthcare parameter;
wherein the first input data request requires the first service application to inform about a first input data information;
receiving from the first personalized healthcare application the first input data information in the interface application, the first input data information being indicative of a data specification of first input data required by the first service application for determining the first personalized healthcare parameter, wherein the data specification specifies in which respective unit(s) of value the first input are required by the first service application;
receiving from the interface application the first input data information in the healthcare application;
generating first personalized input data in the healthcare application, the first personalized input data being generated according to the first input data information, wherein the generation of first personalized input data comprises receiving healthcare data from one or more healthcare data sources and generating the first personalized input data from the received healthcare data which includes checking whether the received healthcare data are complying with the data specification of first input data of the first service application;
receiving the first personalized healthcare parameter in the healthcare application, wherein the first personalized healthcare parameter was determined in response to receiving the first personalized input data in the first service application, such determining comprising processing the first personalized input data; and
outputting the first personalized healthcare parameter to a receiving device connected to the data processing component.

2. The method of claim 1, wherein the generating of the first input data request further comprises, in the interface application,
determining one or more pre-selected first service applications from the plurality of service applications, each of the one or more pre-selected first service applications being configured to determine the first personalized healthcare parameter; and
selecting the first service application from the one or more pre-selected first service applications.

3. The method of claim 2, further comprising determining, by the interface application, the one or more pre-selected first service applications from at least one of:
a first plurality of service applications in a local database locally accessible by and assigned to the interface application; and
a second plurality of service applications remotely accessible in a remote database.

4. The method of claim 1, wherein the submitting of the service request further comprises:
providing service application information with the service request, the service application information being indicative of a selection of the first service application; and
in response to the service application information, generating the first input data request by the interface application, and providing the first input data request to the first service application.

5. The method of claim 1, wherein the generation of first personalized input data further comprises, in the healthcare application,
receiving the healthcare data from one or more healthcare data sources selected from the following group of healthcare data sources: unstructured healthcare database, clinical healthcare database, and regulated healthcare device data.

6. The method of claim 1, wherein the generation of first personalized input data from the received healthcare data in case of non-compliance further comprises converting at least some of the received healthcare data provided with a source data specification into the first personalized input data provided with the data specification of first input data.

7. The method of claim 5, further comprising, in the healthcare application,
converting the first personalized healthcare parameter having an output data specification to the first personalized healthcare parameter having a first source data specification required by a first healthcare data source from the group of the healthcare data sources; and
storing the first personalized healthcare parameter having the first source data specification in the first healthcare data source.

8. The method of claim 1, wherein the submitting of the service request further comprises generating the service request in the healthcare application in response to a trigger event detected in the healthcare application.

9. The method of claim 8, wherein the trigger event is indicative of a user input received in the healthcare application.

10. The method of claim 8, wherein the trigger event is indicative of a trigger point in time.

11. The method of claim 8, wherein the trigger event is indicative of a parameter value change for a medical parameter applied for determining a former first personalized healthcare parameter by means of the first service application, thereby, updating the former first personalized healthcare parameter with the first personalized healthcare parameter.

12. The method of claim 1, further comprising:
receiving the first personalized input data by the interface application;
verifying, based on the first input data information received in the interface application, whether the first personalized input data are complying with the first input data information by the interface application; and
providing the first personalized input data from the interface application to the first service application, only if the first personalized input data are verified to comply with the first input data information.

13. The method of claim 1, wherein the outputting of the first personalized healthcare parameter further comprises:
receiving the first personalized healthcare parameter by a medical device; and
controlling operation of the medical device according to the first personalized healthcare parameter.

14. The method of claim 1, wherein the plurality of service applications comprises service applications configured to determine, for the patient, one or more personalized healthcare parameters selected from the following group: risk prediction, therapy management, and treatment recommendation.

15. A healthcare management system comprising a data processing component, the healthcare management system being configured to:
provide a healthcare application and an interface application running in the data processing component;
submit, from the healthcare application to the interface application, a service request indicative of a request for determining a first personalized healthcare parameter, the first personalized healthcare parameter being indicative of a first healthcare condition of a patient;
in response to receiving the service request, generate a first input data request by the interface application;
provide the first input data request to a first service application from a plurality of service applications, wherein
each of the service applications is configured to determine, for a patient, a respective personalized healthcare parameter in response to receiving respective personalized input data, and
the first service application is configured to determine the first personalized healthcare parameter;
wherein the first input data request requires the first service application to inform about a first input data information;
receive from the first personalized healthcare application first input data information in the interface application, the first input data information being indicative of a data specification of first input data required by the first service application for determining the first personalized healthcare parameter, wherein the data specification specifies in which respective unit(s) of value the first input are required by the first service application;
receive from the interface application the first input data information in the healthcare application;
generate first personalized input data in the healthcare application, the first personalized input data being generated according to the first input data information, wherein the generation of first personalized input data comprises receiving healthcare data from one or more healthcare data sources and generating the first personalized input data from the received healthcare data which includes checking whether the received healthcare data are complying with the data specification of first input data of the first service application;
receive the first personalized healthcare parameter in the healthcare application, wherein the first personalized healthcare parameter was determined in response to receiving the first personalized input data in the first service application, such determining comprising processing the first personalized input data; and
output the first personalized healthcare parameter to a receiving device connected to the data processing component.
